(19) United States
(12) Patent Application Publication
(10) Pub. No.: US 2009/0101140 A1
(43) Pub. Date: Apr. 23, 2009

(54) AIRWAY DEVICES AND METHODS OF MAKING AND USING THE SAME

(76) Inventors: Patrick John Miller, Covington, GA (US); Michael D. Hall, Conyers, GA (US)

Correspondence Address: WITHERS & KEYS, LLC, P. O. BOX 2049, MCDONOUGH, GA, 30253, US

(21) Appl. No.: 12/104,075

(22) Filed: Apr. 16, 2008

Related U.S. Application Data
(60) Provisional application No. 60/923,548, filed on Apr. 16, 2007.

(51) Int. Cl. A61M 16/04

(52) U.S. Cl. 128/200.26

(57) ABSTRACT

Airway devices are disclosed. Methods of making and using airway devices are also disclosed.

### CROSS-REFERENCE TO RELATED APPLICATIONS

This patent application claims the benefit of priority to U.S. Provisional Patent Application Ser. No. 60/923,548 filed on Apr. 16, 2007 and entitled “SUPRAGLOTTIC LARYNGOPHARYNGEAL AIRWAY TUBE”, the subject matter of which is hereby incorporated by reference in its entirety.

### TECHNICAL FIELD

The present invention relates to airway devices including devices referred to as supraglottic airway laryngopharyngeal tubes (SALTs) or oropharyngeal airway tubes. The present invention further relates to methods of making and using airway devices.

### BACKGROUND OF THE INVENTION

Oropharyngeal airways are designed to provide an airway for patients who are unconscious or comatose and are unable to maintain an airway on their own because of an unintact gag reflex. An oropharyngeal airway is inserted into the patient's oropharynx and restrains the tongue from retracting and occluding the glottic opening.

The traditional procedure of endotracheal intubation is typically accomplished in an emergency setting by visualizing the glottic opening with the utilization of a laryngoscope, and then advancing an endotracheal tube through the glottic opening. In the emergency setting, obstacles such as vomitus, blood, or patient positioning can make visualization of the glottic opening extremely difficult if not impossible. Even when aggressive oropharyngeal suctioning is applied, visualization of the glottic opening often fails to be accomplished. If a patient's airway cannot be rapidly and effectively secured, the patient will become hypoxic, which results in rapid deterioration of the patient's health and often results in death.

Given the need for quick action and the difficulty, in some cases, of being able to visually detect the glottic opening of a patient, a number of devices have been developed that do not require visualization of the glottic opening. Such devices have existed for years, but still suffer from one or more drawbacks. For example, the tracheal guide disclosed in U.S. Pat. No. 5,720,275 comprises a distal end in the form of a shovel-like tongue (25) and ears (18,33) positioned along opposite sides of a U-shaped passage (17). In order to operate correctly, the disclosed tracheal guide must be precisely positioned within a patient so that ears (18,33) extend into the piriform fossa (19,34) of the patient, the piriform fossa (19,34) being located in the vicinity of the glottic opening (39) as shown in FIGS. 1-4. In many emergency situations, such precise positioning is not practical given the state of the patient and the need to act quickly.

What is needed in the art is a simple airway device that is (1) capable of quickly aligning certain anatomical structures of a patient's airway so as to provide a guided pathway for an endotracheal tube to be inserted through the device and guided into the trachea of the patient, and (2) can be inserted into a patient without the need for precise positioning of device components/features.

### SUMMARY OF THE INVENTION

The present invention is directed to airway devices that are capable of quickly aligning certain anatomical structures of a patient's airway so as to provide a guided pathway for an endotracheal tube to be inserted through the device and guided into the trachea of the patient. The disclosed devices can be inserted into a patient without the need for precise positioning of device components/features during insertion of the device.

In one exemplary embodiment, the present invention is directed to an airway device comprising a tubular member having a proximal end, a distal end opposite the proximal end, a tubular conduit positioned between the proximal end and the distal end, and a channel extending from a first channel opening at the proximal end through the tubular conduit to a second channel opening proximate the distal end. In this exemplary embodiment, the distal end of the device has an overall distal end width bound by opposing side walls, an overall distal end height bound by an uppermost distal end surface and a lower distal end surface, and a tear-drop shape represented by the uppermost distal end surface, the lower distal end surface, and a curved distal end surface connecting the uppermost distal end surface to the lower distal end surface, wherein the curved distal end surface extends substantially perpendicular to and between the opposing side walls. The tear-drop shape and outer dimensions of the device enable quick insertion of the device into a patient's mouth until the curved distal end surface of the device abuts corniculate cartilage of the patient.

In a further exemplary embodiment, the present invention is directed to an airway device comprising a tubular member having a proximal end, a distal end opposite the proximal end, a tubular conduit positioned between the proximal end and the distal end, and a channel extending from a first channel opening at the proximal end through the tubular conduit to a second channel opening proximate the distal end; and an epiglottis guard extending along an upper portion of the tubular member. In this exemplary embodiment, the epiglottis guard comprises (i) a first end that is connected to the tubular member proximate the second channel opening, (ii) a second end that is not connected to the tubular member and is positioned between the second channel opening and the distal end, and (iii) opposing edges extending from the first end to the second end, wherein the opposing edges are not connected to the tubular member. In this exemplary embodiment, the second end of the epiglottis guard is operatively adapted to move into or away from the channel, for example, during insertion of an endotracheal tube through the channel of the device and into a patient's trachea.

The present invention is further directed to methods of making airway devices suitable for use in an endotracheal intubation procedure. In one exemplary embodiment, the present invention is directed to a method of making an airway device comprising forming a tubular member having a proximal end, a distal end opposite the proximal end, a tubular conduit positioned between the proximal end and the distal end, and a channel extending from a first channel opening at the proximal end through the tubular conduit to a second channel opening proximate the distal end, wherein the distal end of the device has an overall distal end width bound by opposing side walls, an overall distal end height bound by an uppermost distal end surface and a lower distal end surface, and a tear-drop shape represented by the uppermost distal end surface, the lower distal end surface, and a curved distal end surface connecting the uppermost distal end surface to the lower distal end surface, wherein the curved distal end surface extends substantially perpendicular to and between the opposing side walls. In this exemplary embodiment, the forming step may comprise a single thermoforming step (e.g., a single molding step) or may comprise a single thermoforming step in combination with other possible method steps.

In a further exemplary embodiment, the present invention is directed to a method of making an airway device comprising forming a tubular member having a proximal end, a distal end opposite the proximal end, a tubular conduit positioned between the proximal end and the distal end, a channel extending from a first channel opening at the proximal end through the tubular conduit to a second channel opening proximate the distal end, and an epiglottis guard extending along an upper portion of the tubular member. In this exemplary embodiment, the forming step may comprise a single thermoforming step (e.g., a single molding step) or may comprise a thermoforming step (e.g., for forming the tubular member) in combination with one or more other method steps. For example, the epiglottis guard may be formed by cutting an upper portion of the tubular member extending over the channel so as to form an epiglottis guard comprising (i) a first end that is connected to the tubular member proximate the second channel opening, (ii) a second end that is not connected to the tubular member and is positioned between the second channel opening and the distal end, and (iii) opposing cut edges extending from the second end to the first end, wherein the second end of the epiglottis guard is operatively adapted to move into or away from the channel, for example, during insertion of an endotracheal tube through the channel of the device and into a patient's trachea.

The present invention is even further directed to methods of using airway devices in an endotracheal intubation procedure. In one exemplary embodiment, the present invention is directed to a method of inserting an endotracheal tube into a trachea of a patient comprising the steps of inserting an airway device into the patient's mouth until a curved distal end surface of the device abuts corniculate cartilage of the patient, the device comprising a tubular member having a proximal end, a distal end opposite the proximal end, a tubular conduit positioned between the proximal end and the distal end, and a channel extending from a first channel opening at the proximal end through the tubular conduit to a second channel opening proximate the distal end, wherein the distal end has an overall distal end width bound by opposing side walls, an overall distal end height bound by an uppermost distal end surface and a lower distal end surface, and a tear-drop shape represented by the uppermost distal end surface, the lower distal end surface, and the curved distal end surface connecting the uppermost distal end surface to the lower distal end surface, the curved distal end surface extending substantially perpendicular to and between the opposing side walls; and pushing an endotracheal tube through the channel of the device. This exemplary method may comprise one or more additional steps including, but not limited to, connecting a ventilation mask to the proximate end of the device after the inserting step, disconnecting the ventilation mask from the proximate end of the device after the connecting step and prior to said pushing step, and coating at least a portion of a leading end of the endotracheal tube with a lubricant prior to the pushing step.

The present invention is even further directed to kits suitable for performing an endotracheal intubation procedure. In one exemplary embodiment, the kit comprises at least one of the disclosed airway devices in combination with an endotracheal tube. The kits of the present invention may further comprise additional kit components including, but not limited to, a lubricant, and a ventilation mask.

These and other features and advantages of the present invention will become apparent after a review of the following detailed description of the disclosed embodiments and the appended claims.

### BRIEF DESCRIPTION OF THE DRAWINGS

The present invention is further described with reference to the appended figures, wherein:

FIG. 1 provides a perspective view of an exemplary airway device of the present invention;

FIG. 2 provides a side view of the exemplary airway device shown in FIG. 1;

FIG. 3 provides a top view of the exemplary airway device shown in FIG. 1;

FIG. 4 provides a side view of the exemplary airway device shown in FIG. 1 with an endotracheal tube inserted through the exemplary airway device;

FIG. 5 provides a view of the exemplary airway device shown in FIG. 1 positioned within a patient during an endotracheal intubation procedure;

FIG. 6 provides a top view of another exemplary airway device of the present invention;

FIG. 7 provides a side view of the exemplary airway device shown in FIG. 6;

FIG. 8 provides a cross-sectional view of the exemplary airway device shown in FIG. 6 along line B-B shown in FIG. 6;

FIG. 9 provides a rear view of a distal end of the exemplary airway device shown in FIG. 6; and

FIG. 10 provides a view of the exemplary airway device shown in FIG. 6 positioned within a patient during an endotracheal intubation procedure.

### DETAILED DESCRIPTION OF THE INVENTION

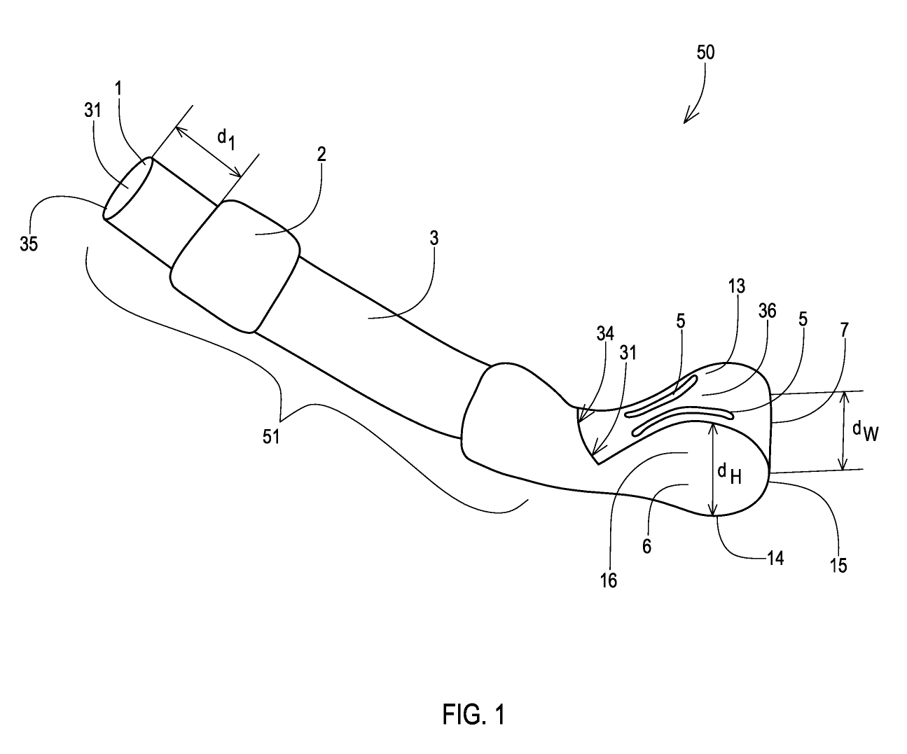

The present invention is directed to airway devices suitable for use in an endotracheal intubation procedure. As used herein, the phrase “airway device” or “airway devices” is used to describe a class of devices that includes, but is not limited to, supraglottic airway laryngopharyngeal tubes (SALTs) and oropharyngeal airway tubes. In particular, the disclosed airway devices are suitable for use in procedures for providing ventilation (e.g., air) to a patient, procedures for inserting an endotracheal tube into the trachea of a patient, or both. The present invention is further directed to methods of making airway devices, as well as methods of using airway devices in an endotracheal intubation procedure. One exemplary airway device of the present invention suitable for use in an endotracheal intubation procedure is shown as exemplary airway device 50 in FIG. 1.

Referring to FIG. 1, exemplary airway device 50 comprises a tubular member 51 having a proximal end 1, a distal end 6 opposite proximal end 1, a tubular conduit 3 positioned between proximal end 1 and distal end 6, and a channel 31 extending from a first channel opening 35 at proximal end 1 through tubular conduit 3 to a second channel opening 34 proximate distal end 6. Exemplary airway device 50 further comprises a depth indicator ring 2 proximate proximal end 1. Although shown positioned a distance d₁ from proximal end 1, it should be understood that depth indicator ring 2 may be positioned at proximal end 1 (i.e., d₁=0) or any distance d₁ from proximal end 1. Typically, depth indicator ring 2 has an outer diameter that is greater than an outer diameter of tubular conduit 3 as shown in FIG. 1. As discussed further below, depth indicator ring 2 may be used to signal a proper depth of insertion into a patient's mouth during an endotracheal intubation procedure.

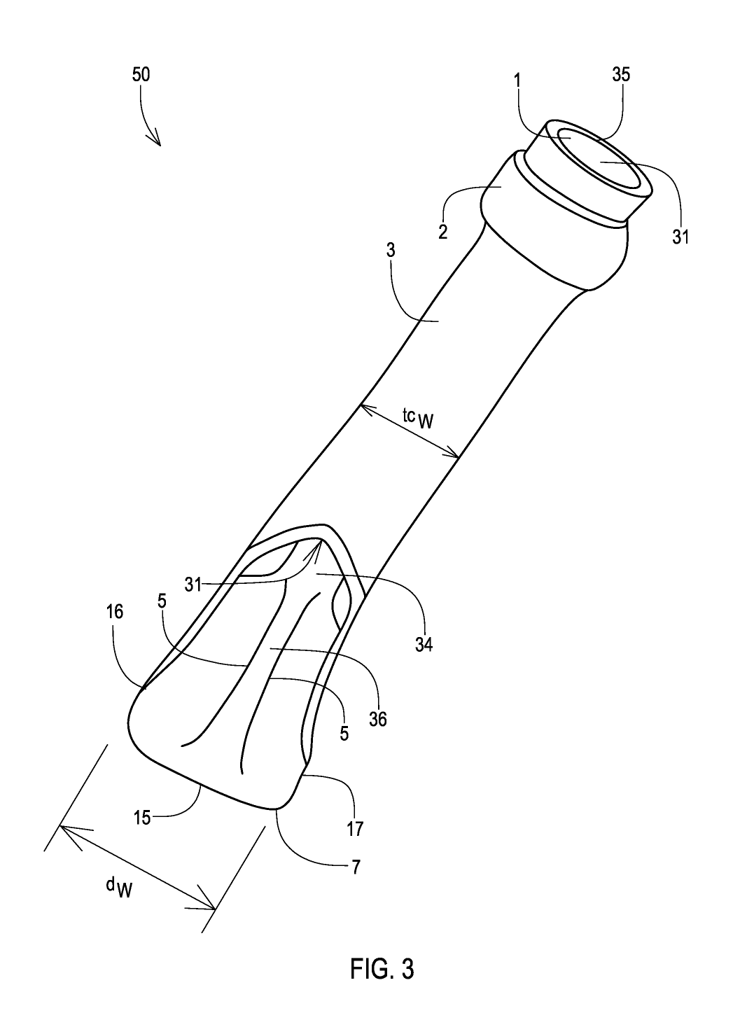

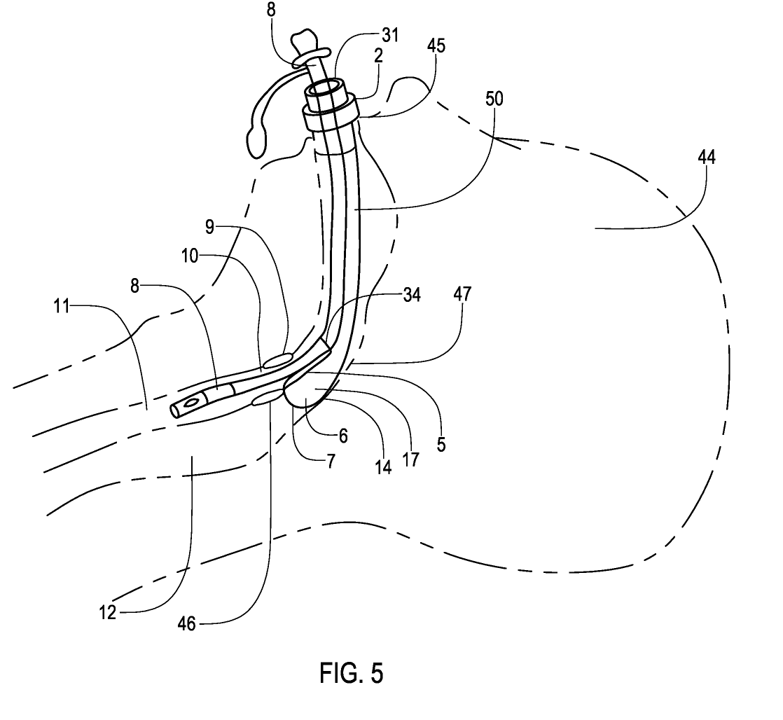

As shown in FIG. 1, distal end 6 of exemplary airway device 50 has an overall distal end width, d_{W}, bound by opposing side walls 16 and 17 (see, FIG. 3), an overall distal end height, d_{H}, bound by an uppermost distal end surface 13 and a lower distal end surface 14, and a tear-drop shape represented by uppermost distal end surface 13, lower distal end surface 14, and a curved distal end surface 15 connecting uppermost distal end surface 13 to lower distal end surface 14. As shown in FIG. 1, curved distal end surface 15 extends substantially perpendicular to and between opposing side walls 16 and 17. In one desired embodiment, at least a portion of each of opposing side walls 16 and 17 (see, FIG. 3) proximate curved distal end surface 15 extends substantially parallel to one another so as to form right angles with curved distal end surface 15. As discussed further below (and shown in FIG. 5), the tear-drop shape and outer dimensions of exemplary airway device 50 enable quick insertion of exemplary airway device 50 into a patient's mouth until curved distal end surface 15 of exemplary airway device 50 abuts corniculate cartilage of a patient.

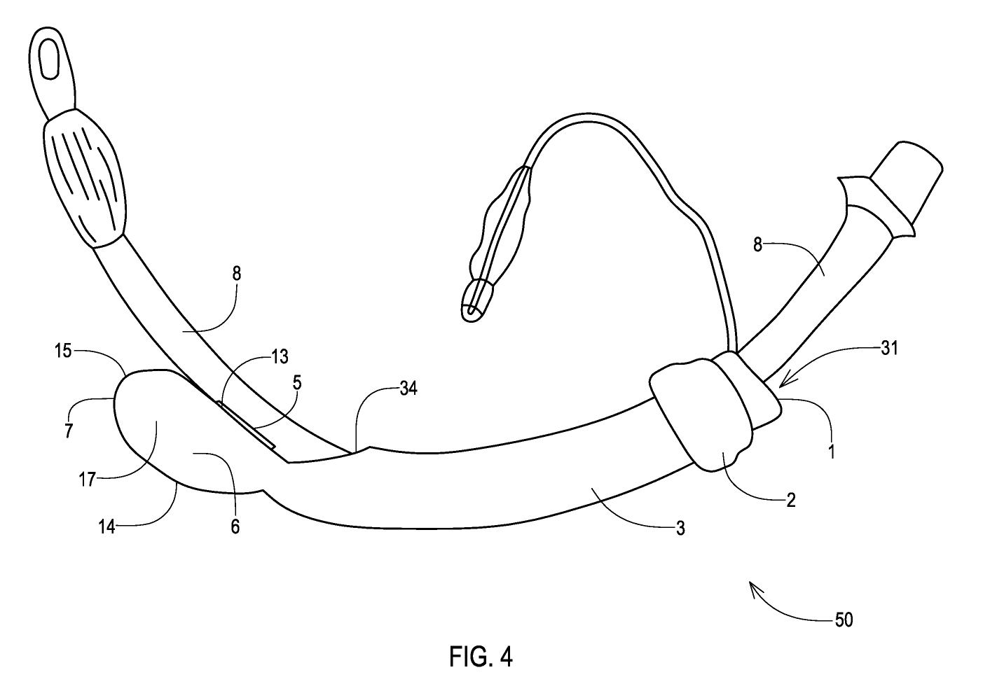

In some exemplary embodiments, the airway devices of the present invention further comprise a pair of raised ridges that form a channel extension operatively adapted to direct an endotracheal tube along an uppermost distal end surface of the device toward a glottic opening of a patient. As shown in FIG. 1, exemplary airway device 50 comprises raised ridges 5 extending along uppermost distal end surface 13 between second channel opening 34 and a tip portion 7 of distal end 6. Raised ridges 5 form a channel extension 36 that is operatively adapted to direct an endotracheal tube (not shown) along uppermost distal end surface 13 toward a glottic opening of a patient (see, FIGS. 4-5).

Desirably, raised ridges 5 are mirror images of one another. In other words, a line dissecting channel extension 36 would be equally spaced from corresponding points along each of raised ridges 5. In some embodiments, raised ridges 5 are substantially parallel with one another. In other embodiments, such as shown in FIG. 1, raised ridges 5 have some curvature therein. For example, raised ridges 5 may be configured such that a shortest distance between raised ridges 5 is along a center portion of a given raised ridge 5 or along a portion of a given raised ridge 5 proximate second channel opening 34 while a greatest distance between raised ridges 5 is along a portion of a given raised ridge 5 proximate tip portion 7 of distal end 6 (see, for example, raised ridge 5 shown in FIG. 3).

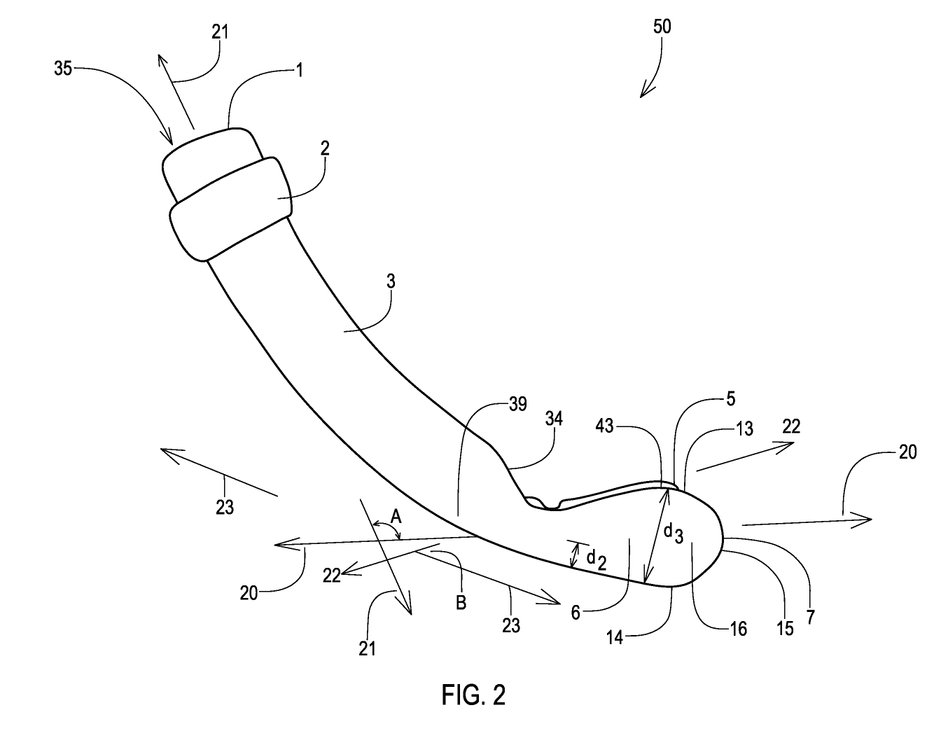

As shown in FIG. 2, exemplary airway device 50 has a curved configuration. The curved configuration may be further described with reference to lines 20-20 and 21-21, as well as angle A formed therebetween. In one desired embodiment, tubular member 51 has a curved section between proximal end 1 and distal end 6 such that (i) a first line 21-21 extending substantially parallel to tubular member 51 out of first channel opening 35 (i.e., dissects first channel opening 35) and (ii) a second line 20-20 extending from distal end 6 through curved distal end surface 15 and positioned an equal distance from uppermost distal end surface 13 and lower distal end surface 14 forms an angle A with one another of less than 180°. Typically, angle A ranges from about 110° to about 165°, more typically, from about 130° to about 145°, and in one exemplary embodiment, about 135°.

Further, as shown in FIG. 2, distal end 6 of exemplary airway device 50 desirably provides an upward surface inclination from second channel opening 34 to a point along uppermost distal end surface 13. In one desired embodiment, channel 31 has a lowest channel point 39 along channel 31, wherein lowest channel point 39 is a first distance d₂ above lower distal end surface 14, and channel extension 36 has a highest channel extension point 43 along uppermost distal end surface 13, wherein highest channel extension point 43 is a second distance d₃ above lower distal end surface 14, wherein second distance d₃ is greater than first distance d₂. Desirably, highest channel extension point 43 comprises an uppermost point along uppermost distal end surface 13.

This surface inclination feature of exemplary airway device 50 may be further described with reference to lines 22-22 and 23-23, as well as angle B formed therebetween. In one desired embodiment, distal end 6 of exemplary airway device 50 provides an upward surface inclination such that (i) first line 23-23 extending substantially parallel to lower distal end surface 14 and (ii) line 22-22 extending through lowest channel point 39 and along uppermost distal end surface 13 forms an angle B with one another of greater than about 10°. Typically, angle B ranges from about 10° to about 60°, more typically, from about 25° to about 50°, and in one exemplary embodiment, about 40°.

FIG. 3 provides a top view of exemplary airway device 50. In FIG. 3, channel 31, first channel opening 35, second channel opening 34, raised ridges 5, channel extension 36, and opposing side walls 16 and 17 are more depicted. As shown in FIG. 3, overall distal end width d_{W} is typically greater than an overall width tc_{W} of tubular conduit 3. As discussed further below, overall distal end width d_{W} functions to seat exemplary airway device 50 against corniculate cartilage of a patient when positioned within the patient's oropharnyx without any portion of exemplary airway device 50 extending into the esophagus or piriform fossa of the patient.

Exemplary airway device 50 may be used in combination with an endotracheal tube during an endotracheal intubation procedure. FIG. 4 provides a side view of exemplary airway device 50 in combination with an endotracheal tube 8 inserted through channel 31 of exemplary airway device 50. As shown in FIG. 4, endotracheal tube 8 extends through channel 31 of exemplary airway device 50, out of second channel opening 34 and along channel extension 36 between raised ridges 5 along uppermost distal end surface 13. FIG. 5 provides a view of exemplary airway device 50 in combination with endotracheal tube 8 as positioned within a patient 44 during an endotracheal intubation procedure.

As shown in FIG. 5, exemplary airway device 50 extends from out of mouth 45 of patient 44 to corniculate cartilage 46 proximate the opening into the esophagus 12 of patient 44. Distal end tip 7 of distal end 6 abuts corniculate cartilage 46 so as to position second channel opening 34 proximate glottic opening 10 leading into the trachea 11 of patient 44. Overall distal end height, d_{H}, of distal end 6 prevents exemplary airway device 50 from extending beyond corniculate cartilage 46 and into esophagus 12. (Note that lower distal end surface 14 is in contact with throat wall 47 so as to lodge distal end 6 against corniculate cartilage 46.) Endotracheal tube 8 extends through channel 31 of exemplary airway device 50, out of second channel opening 34, along channel extension 36 between raised ridges 5 and along uppermost distal end surface 13, through glottic opening 10 and into trachea 11 of patient 44.

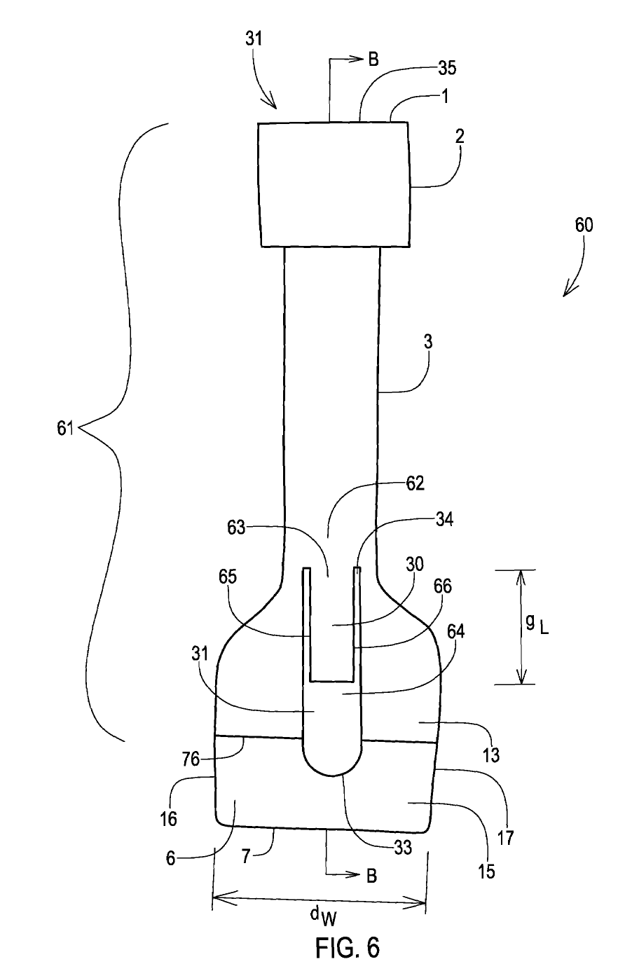

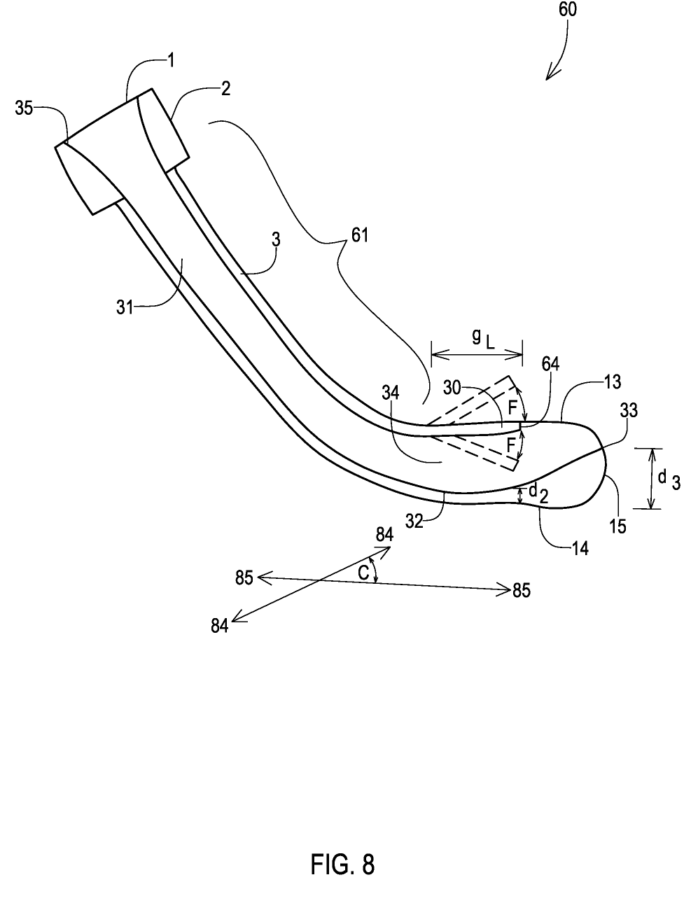

FIG. 6 provides a top view of another exemplary airway device 60 of the present invention. As shown in FIG. 6, exemplary airway device 60 comprises tubular member 61 having a proximal end 1, a distal end 6 opposite proximal end 1, a tubular conduit 3 positioned between proximal end 1 and distal end 6, and a channel 31 extending from a first channel opening 35 at proximal end 1 through tubular conduit 3 to a second channel opening 34 proximate distal end 6. Exemplary airway device 60 further comprises an epiglottis guard 30 extending along an upper portion 62 of tubular member 61, wherein epiglottis guard 30 comprises (i) a first end 63 that is connected to tubular member 61 proximate second channel opening 34, (ii) a second end 64 that is not connected to tubular member 61 and is positioned between second channel opening 34 and distal end 6, and (iii) opposing edges 65 and 66 extending from first end 63 to second end 64, wherein opposing edges 65 and 66 are not connected to tubular member 61, and second end 64 is operatively adapted to move into or away from channel 31 (see, for example, possible movement of second end 64 as shown in FIG. 8).

Epiglottis guard 30 has a guard length, g_{L}, extending the length of opposing edges 65 and 66. Guard length, g_{L}, may vary as desired, but typically guard length, g_{L}, extends a distance that is less than or equal to a distance from proximate second channel opening 34 to an end point 33 of channel 31. More typically, guard length, g_{L}, extends a distance that is about half the distance from proximate second channel opening 34 to end point 33 of channel 31. In dimensions, guard length, g_{L}, typically ranges from about 0.5 inches to about 1.5 inches in length.

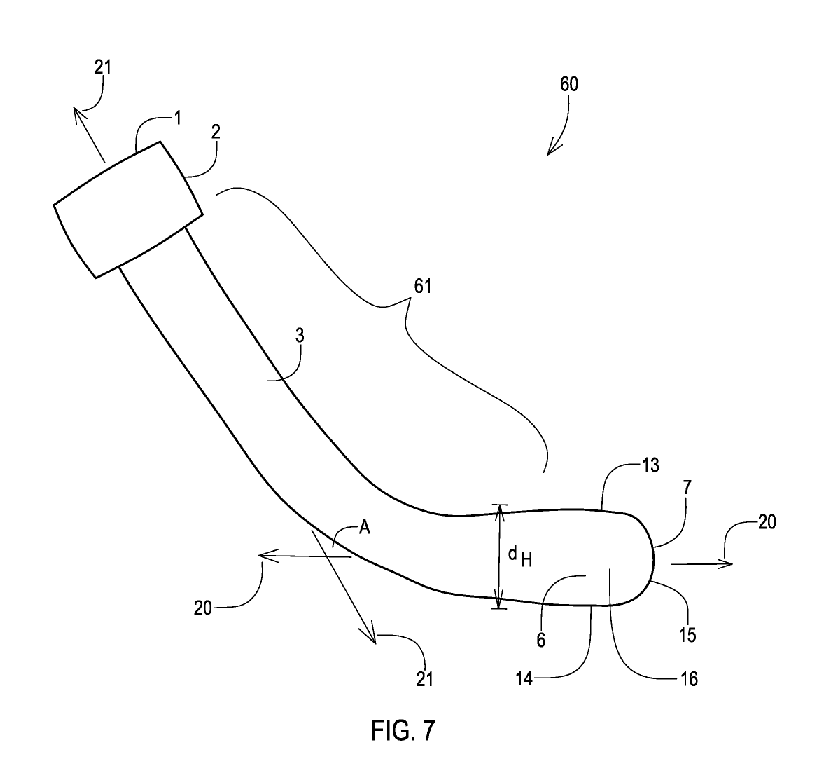

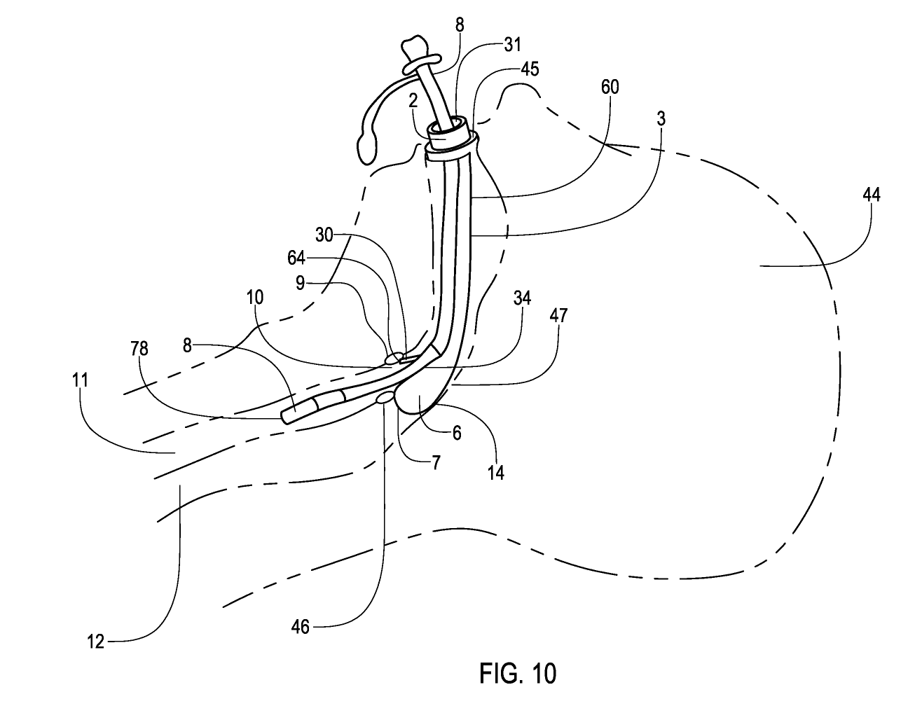

As shown in FIG. 6, distal end 6 of exemplary airway device 60 has an overall distal end width, d_{W}, bound by opposing side walls 16 and 17. As shown in FIG. 7, distal end 6 of exemplary airway device 60 has an overall distal end height, d_{H}, bound by uppermost distal end surface 13 and lower distal end surface 14, and a tear-drop shape represented by uppermost distal end surface 13, lower distal end surface 14, and a curved distal end surface 15 connecting uppermost distal end surface 13 to lower distal end surface 14. (The intersection of uppermost distal end surface 13 and curved distal end surface 15 being depicted as junction 76.) Desirably, curved distal end surface 15 extends substantially perpendicular to and between opposing side walls 16 and 17 (see, FIG. 6). Similar to exemplary airway device 50 discussed above, the tear-drop shape and outer dimensions of exemplary airway device 60 enable quick insertion of exemplary airway device 60 into a patient's mouth until curved distal end surface 15 of exemplary airway device 60 abuts corniculate cartilage of a patient (as shown in FIG. 10).

FIG. 7 provides a side view of exemplary airway device 60 shown in FIG. 6. As shown in FIG. 7, exemplary airway device 60 desirably has a curved configuration. The curved configuration may be further described with reference to lines 20-20 and 21-21, as well as angle A formed therebetween. In one desired embodiment, tubular member 61 has a curved section between proximal end 1 and distal end 6 such that (i) a first line 21-21 extending substantially parallel to tubular member 61 out of first channel opening 35 (i.e., dissects first channel opening 35) and (ii) a second line 20-20 extending from distal end 6 through curved distal end surface 15 and positioned an equal distance from uppermost distal end surface 13 and lower distal end surface 14 forms an angle A with one another of less than 180°. Typically, angle A ranges from about 110° to about 165°, more typically, from about 130° to about 145°, and in one exemplary embodiment, about 140°.

FIG. 8 provides a cross-sectional view of exemplary airway device 60 shown in FIG. 6 along line B-B shown in FIG. 6. As shown in FIG. 8, channel 31 extends from first channel opening 35 along tubular member 61 through second channel opening 34 to end point 33. As shown in FIG. 8, first channel opening 35 may have an opening inner diameter slightly larger than an inner diameter of tubular conduit 3.

Further, as shown in FIG. 8, distal end 6 of exemplary airway device 60 desirably provides an upward surface inclination from second channel opening 34 to a point along uppermost distal end surface 13. In one desired embodiment, channel 31 has a lowest channel point 32 along channel 31, wherein lowest channel point 32 is a first distance d₂ above lower distal end surface 14, and channel 31 has a highest channel point at end point 33 along uppermost distal end surface 13 or curved distal end surface 15, wherein end point 33 is a second distance d₃ above lower distal end surface 14, wherein second distance d₃ is greater than first distance d₂.

This feature of exemplary airway device 60 may be further described with reference to lines 84-84 and 85-85, as well as angle C formed therebetween. In one desired embodiment, distal end 6 of exemplary airway device 60 provides an upward surface inclination such that (i) first line 85-85 extending substantially parallel to lower distal end surface 14 and (ii) line 84-84 extending through lowest channel point 32 and through end point 33 forms an angle C with one another of greater than about 10°. Typically, angle C ranges from about 10° to about 60°, more typically, from about 25° to about 50°, and in one exemplary embodiment, about 40°.

As shown in FIG. 8 and discussed above, second end 64 of epiglottis guard 30 is operatively adapted to move into or away from channel 31 as designated by up and down arrows F. As discussed further below with reference to FIG. 10, epiglottis guard 30 prevents the epiglottis of a patient from blocking the glottic opening during an endotracheal intubation procedure.

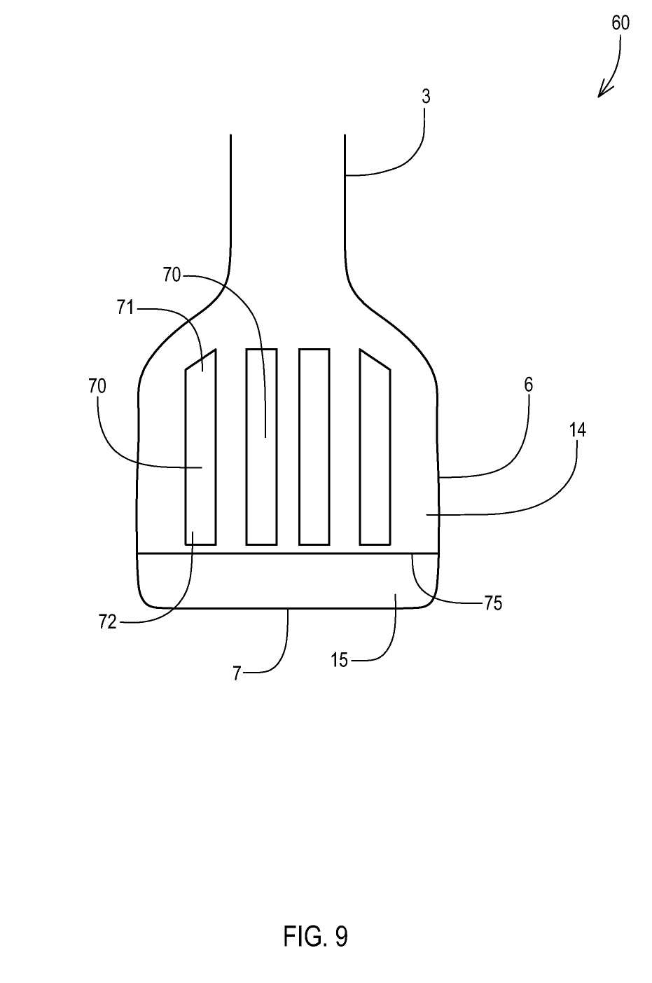

FIG. 9 provides a rear view of distal end 6 of exemplary airway device 60 shown in FIG. 6. As shown in FIG. 9, lower distal end surface 14 may have one or more fluid distribution channels 70 extending along a length of lower distal end surface 14. Typically, fluid distribution channels 70 are oriented so as to extend from a location proximate second channel opening 34 to a location proximate a junction 75 between lower distal end surface 14 and curved distal end surface 15. Each of fluid distribution channels 70 may have dimensions that vary depending on the outer dimensions of distal end 6. A depth of a given fluid distribution channel 70 (i.e., the dimension extending into the page) may be constant along a length of fluid distribution channel 70 or may vary along a length of fluid distribution channel 70. In one exemplary embodiment, the depth of each of the fluid distribution channels 70 increases from location 71 to location 72 within a given fluid distribution channel 70 so as to follow along the inclination angle of channel 31 (e.g., angle C shown in FIG. 8). In another exemplary embodiment, the depth of each of the fluid distribution channels 70 is substantially the same from location 71 to location 72 within a given fluid distribution channel 70.

It should be understood that fluid distribution channels 70 are one optional feature for distal end 6 of exemplary airway device 50 and/or 60, and are not required. The presence of fluid distribution channels 70 may provide one or more advantages (i) to the resulting device (e.g., enhanced structural stability at the distal end), (ii) during use (e.g., enhanced fluid flow within a patient's throat), as well as (iii) during manufacturing (e.g., reduces mold element thickness of any given portion of the distal end using an injection molding step).

FIG. 10 provides a view of exemplary airway device 60 in combination with endotracheal tube 8 as positioned within a patient 44 during an endotracheal intubation procedure. As shown in FIG. 10, exemplary airway device 60 extends from out of mouth 45 of patient 44 to corniculate cartilage 46 proximate the opening into the esophagus 12 of patient 44. Distal end tip 7 of distal end 6 abuts corniculate cartilage 46 so as to position second channel opening 34 and epiglottis guard 30 proximate glottic opening 10 leading into the trachea 11 of patient 44. Overall distal end height, d_{H}, of distal end 6 prevents exemplary airway device 60 from extending beyond corniculate cartilage 46 and into esophagus 12. (Note that lower distal end surface 14 is in contact with throat wall 47 so as to lodge distal end 6 against corniculate cartilage 46.) Endotracheal tube 8 extends through channel 31 of exemplary airway device 60, out of second channel opening 34, along channel 31 and along uppermost distal end surface 13, through glottic opening 10 and into trachea 11 of patient 44.

As shown in FIG. 10, epiglottis guard 30 of exemplary airway device 60 comes into contact with epiglottis 9 when endotracheal tube 8 extends out of second channel opening 34. As leading end 78 of endotracheal tube 8 moves out of second channel opening 34 and along uppermost distal end surface 13, the outer dimensions of endotracheal tube 8 exert an upward force on second end 64 of epiglottis guard 30, causing second end 64 of epiglottis guard 30 to push epiglottis 9 out of glottic opening 10 so that leading end 78 of endotracheal tube 8 can move into trachea 11 of patient 44 without obstruction.

The present invention is further directed to methods of making airway devices suitable for use in an endotracheal intubation procedure. In one exemplary embodiment, the present invention is directed to a method of making an airway device (e.g., exemplary airway device 50 or 60) comprising forming a tubular member 51 or 61 having a proximal end 1, a distal end 6 opposite proximal end 1, a tubular conduit 3 positioned between proximal end 1 and distal end 6, and a channel 31 extending from a first channel opening 35 at proximal end 1 through tubular conduit 3 to a second channel opening 34 proximate distal end 6, wherein distal end 6 of device 50 or 60 has an overall distal end width, d_{W}, bound by opposing side walls 16 and 17, an overall distal end height, d_{H}, bound by an uppermost distal end surface 13 and a lower distal end surface 14, and a tear-drop shape represented by uppermost distal end surface 13, lower distal end surface 14, and a curved distal end surface 15 connecting uppermost distal end surface 13 to lower distal end surface 14, wherein curved distal end surface 15 extends substantially perpendicular to and between opposing side walls 16 and 17.

In one exemplary embodiment, the forming step comprises a single thermoforming step (e.g., a single injection molding step), wherein thermoformable material is placed into the mold (e.g., injected) and molded to form an airway device (e.g., exemplary airway device 50 or 60). Suitable thermoformable materials for forming the disclosed airway devices include, but are not limited to, polyvinyl chlorides and polyurethanes. In one desired embodiment, the thermoformable material used to form the disclosed airway devices comprises a medical grade polyvinyl chloride.

The method of making an airway device may further comprise one or more additional steps in addition to the thermoforming step. Suitable additional method steps may include, but are not limited to, removing the molded object (e.g., the airway device) from a mold, trimming any excess material from the airway device, coating the airway device with a finish on any outer surface (e.g., coating at least a portion of channel 31 surface with a lubricant or slip agent), and forming a kit containing the airway device and at least one other kit component. Desirably, the method of making an airway device comprises a single thermoforming step (e.g., injection molding) without ant additional steps other than packaging the resulting device.

In a further exemplary embodiment, the method of making an airway device (e.g., exemplary airway device 60) comprises forming a tubular member 61 having a proximal end 1, a distal end 6 opposite proximal end 1, a tubular conduit 3 positioned between proximal end 1 and distal end 6, a channel 31 extending from a first channel opening 35 at proximal end 1 through tubular conduit 3 to a second channel opening 34 proximate distal end 6, and an epiglottis guard 30 extending along an upper portion 62 of tubular member 60. In this exemplary embodiment, the forming step may comprise a single thermoforming step (e.g., a single injection molding step as described above) or may comprise a thermoforming step (e.g., for forming tubular member 61) in combination with one or more other method steps. For example, epiglottis guard 30 may be formed by (i) a single molding step or (ii) in a separate step comprising cutting upper portion 62 of tubular member 61 extending over channel 31 so as to form epiglottis guard 30 comprising (i) first end 63 that is connected to tubular member 61 proximate second channel opening 34, (ii) second end 64 that is not connected to tubular member 61 and is positioned between second channel opening 34 and distal end 6, and (iii) opposing cut edges 65 and 66 extending from second end 64 to first end 63, wherein second end 64 of epiglottis guard 30 is operatively adapted to move into or away from channel 31, for example, during insertion of endotracheal tube 8 through channel 31 of device 60 and into a patient's trachea 11.

In any of the above-described methods of forming an airway device, a single thermoforming step (e.g., molding step) may be used to form one or more fluid distribution channels 70 along lower distal end surface 14 as described above. Any of the above-mentioned additional steps (other than the thermoforming step) may be used to further provide one or more features to the resulting airway device.

The present invention is further directed to methods of using the above-described airway devices (e.g., exemplary airway device 50 or 60) in an endotracheal intubation procedure. In one exemplary embodiment, the method of inserting an endotracheal tube 8 into a trachea 11 of a patient 44 comprises the steps of inserting an airway device (e.g., exemplary airway device 50 or 60) into the patient's mouth 45 until a curved distal end surface 15 of the device abuts corniculate cartilage 46 of the patient 44, wherein (1) the device comprises a tubular member 51 or 61 having a proximal end 1, a distal end 6 opposite proximal end 1, a tubular conduit 3 positioned between proximal end 1 and distal end 6, and a channel 31 extending from a first channel opening 35 at proximal end 1 through tubular conduit 3 to a second channel opening 34 proximate distal end 6, (2) distal end 6 has an overall distal end width, d_{W}, bound by opposing side walls 16 and 17, an overall distal end height, d_{H}, bound by an uppermost distal end surface 13 and a lower distal end surface 14, and a tear-drop shape represented by uppermost distal end surface 13, lower distal end surface 14, and curved distal end surface 15 connecting uppermost distal end surface 13 to lower distal end surface 14, and (3) curved distal end surface 15 extends substantially perpendicular to and between opposing side walls 16 and 17; and pushing endotracheal tube 8 through channel 31 of the device.

The exemplary method of using the above-described airway devices may comprise one or more additional steps including, but not limited to, connecting a ventilation mask (not shown) to the proximal end 1 of the device after the inserting step; disconnecting the ventilation mask from the proximal end 1 of the device after the connecting step and prior to the pushing step; coating at least a portion of a leading end 78 of endotracheal tube 8 with a lubricant (not shown) prior to the pushing step; and any combination of the above-mentioned steps.

The above-described airway devices are desirably free from any inflatable components. In other words, the disclosed airway devices comprise a rigid structure that is not inflatable. Further, the disclosed airway devices are typically free from any voids or empty spaces other than the above-described channel (e.g., channel 31) and the above-described optional fluid distribution channels (e.g., fluid distribution channels 70).

In addition, the above-described tear-drop shape represented by the uppermost distal end surface, the lower distal end surface, and the curved distal end surface connecting the uppermost distal end surface to the lower distal end surface is described as having a curved distal end surface that extends substantially perpendicular to and between the opposing side walls. It should be noted that the curved distal end surface desirably extends substantially perpendicular to and between the opposing side walls continuously along an outer periphery of each of the opposing side walls. In other words, the distal end typically does not contain any voids or empty space between the opposing side walls and along the curved distal end surface except possibly a portion of channel 31. Further, the distal end tip (e.g., tip 7) positioned along the distal end (e.g., distal end 6) does not comprise any further extensions beyond curved distal end surface (e.g., curved distal end surface 15). In other words, curved distal end surface forms an outermost portion of the distal end tip.

Although the above-described airway devices may have any desired dimensions, typically the above-described airway devices have dimensions as shown in the table below.

| Dimension | Typical Range | More Typical Range |
|---|---|---|
| overall length | about 5.0 to about 8.0 in. | about 6.0 to about 7.0 in. |
| distal end width, d_{W} | about 1.0 to about 3.0 in. | about 1.25 to about 1.5 in. |
| distal end height, d_{H} | about 0.5 to about 1.5 in. | about 0.75 to about 1.0 in. |
| channel inner diameter | about 0.5 to about 1.5 in. | about 0.75 to about 1.0 in. |
| tubular conduit outer | about 0.75 to about 2.0 in. | about 1.0 to about 1.5 in. |
| diameter | | |
| length of depth | about 0.75 to about 2.0 in. | about 1.0 to about 1.5 in. |
| indicator ring | | |
| tubular conduit wall | about 0.10 to about 0.25 in. | about 0.17 to about 0.20 in. |
| thickness | | |
| wall thickness of | about 0.10 to about 0.375 in. | about 0.1875 to about 0.25 in. |
| depth indicator ring | | |
| length of epiglottis guard | about 0.5 to about 2.5 in. | about 0.75 to about 1.25 in. |
| angle of curvature | about 110° to about 170° | about 130° to about 150° |
| along device (e.g., | | |
| angle A shown in | | |
| FIGS. 2 and 7) | | |
| angle of channel | about 20° to about 60° | about 30° to about 45° |
| inclination (e.g., | | |
| angle B shown in | | |
| FIG. 2 and angle C | | |
| shown in FIG. 8) | | |

The above-described airway devices may be provided as an individual component or as one component in a kit for performing an endotracheal intubation procedure. One exemplary kit comprises at least one of the above-described airway devices in combination with an endotracheal tube. Another exemplary kit comprises kit components including, but not limited to, at least one of the above-described airway devices, an endotracheal tube, a lubricant, a ventilation mask, or any combination thereof.

The present invention is described above and further illustrated below by way of examples, which are not to be construed in any way as imposing limitations upon the scope of the invention. On the contrary, it is to be clearly understood that resort may be had to various other embodiments, modifications, and equivalents thereof which, after reading the description herein, may suggest themselves to those skilled in the art without departing from the spirit of the present invention and/or the scope of the appended claims.

### EXAMPLE

An airway device was formed via an injection molding step in which medical grade polyvinyl chloride was molded into a shape as shown in FIGS. 6-9. The resulting airway device had the following dimensions:

length=6.5 inches

width of distal end=1.375 inches

height of distal end=0.875 inch

inner diameter=0.875 inch

outer diameter=1.25 inches

length of depth indicator ring=1.125 inches

tubular conduit wall thickness=0.1875 inch

angle of curvature along device=140°.

While the specification has been described in detail with respect to specific embodiments thereof, it will be appreciated that those skilled in the art, upon attaining an understanding of the foregoing, may readily conceive of alterations to, variations of, and equivalents to these embodiments. Accordingly, the scope of the present invention should be assessed as that of the appended claims and any equivalents thereto.

What is claimed is:

1. An airway device comprising:
a tubular member having a proximal end, a distal end opposite the proximal end, a tubular conduit positioned between the proximal end and the distal end, and a channel extending from a first channel opening at the proximal end through the tubular conduit to a second channel opening proximate the distal end; said distal end having an overall distal end width bound by opposing side walls, an overall distal end height bound by an uppermost distal end surface and a lower distal end surface, and a tear-drop shape represented by the uppermost distal end surface, the lower distal end surface, and a curved distal end surface connecting the uppermost distal end surface to the lower distal end surface, said curved distal end surface extending substantially perpendicular to and between said opposing side walls.

2. The device of claim 1, wherein said overall distal end width is greater than an overall width of said tubular conduit.

3. The device of claim 1, wherein said tubular member has a curved section between said proximal end and said distal end such that (i) a first line extending substantially parallel to said tubular member out of said first channel opening and (ii) a second line extending from said distal end through said curved distal end surface and positioned an equal distance from said uppermost distal end surface to and said lower distal end surface forms an angle with one another of less than 180°.

4. The device of claim 3, wherein said angle ranges from about 110° to about 165°.

5. The device of claim 1, further comprising a depth indicator ring positioned along said tubular member proximate said proximal end, said depth indicator ring having an outer diameter that is greater than an outer diameter of said tubular conduit.

6. The device of claim 1, further comprising a pair of raised ridges extending along said uppermost distal end surface between said second channel opening and said distal end, said pair of raised ridges forming a channel extension operatively adapted to direct an endotracheal tube along said uppermost distal end surface toward a glottic opening of a patient.

7. The device of claim 6, wherein said channel has a lowest channel point along said channel, said lowest channel point being a first distance above said lower distal end surface, and said channel extension having a highest channel extension point along said uppermost distal end surface, said highest channel extension point comprises an uppermost point along said being a second distance above said lower distal end surface, said second distance being greater than said first distance.

8. The device of claim 7, wherein said highest channel extension point comprises an uppermost point along said uppermost distal end surface.

9. The device of claim 1, further comprising an epiglottis guard extending along an upper portion of said tubular member, said epiglottis guard comprising (i) a first end that is connected to said tubular member proximate said second channel opening, (ii) a second end that is not connected to said tubular member and positioned between said second channel opening and said distal end, and (iii) opposing edges extending from said first end to said second end, said opposing edges being not connected to said tubular member, wherein said second end is operatively adapted to move into or away from said channel.

10. The device of claim 1, wherein said curved distal end surface of said device provides a rigid structure that is sized so as to abut corniculate cartilage of a patient when positioned within the patient's oropharnyx without any portion of said device extending into the esophagus or piriform fossa of the patient.

11. The device of claim 1, wherein said device is free from any inflatable components.

12. An airway device comprising:
a tubular member having a proximal end, a distal end opposite the proximal end, a tubular conduit positioned between the proximal end and the distal end, and a channel extending from a first channel opening at the proximal end through the tubular conduit to a second channel opening proximate the distal end; and
an epiglottis guard extending along an upper portion of said tubular member, said epiglottis guard comprising (i) a first end that is connected to said tubular member proximate said second channel opening, (ii) a second end that is not connected to said tubular member and positioned between said second channel opening and said distal end, and (iii) opposing edges extending from said first end to said second end, said opposing edges being not connected to said tubular member, wherein said second end is operatively adapted to move into or away from said channel.

13. The device of claim 12, wherein said distal end has an overall distal end width bound by opposing side walls, an overall distal end height bound by an uppermost distal end surface and a lower distal end surface, and a tear-drop shape represented by the uppermost distal end surface, the lower distal end surface, and a curved distal end surface connecting the uppermost distal end surface to the lower distal end surface, said curved distal end surface extending substantially perpendicular to and between said opposing side walls.

14. The device of claim 13, wherein said lower distal end surface further comprises a plurality of fluid distribution channels extending therein along a length of said lower distal end surface.

15. A kit comprising:
the device of claim 12; and
an endotracheal tube.

16. The kit of claim 15, further comprising at least one of:
a lubricant; and
a ventilation mask.

17. A method of inserting an endotracheal tube into a trachea of a patient, said method comprising the steps of:
inserting the device of claim 12 into the patient's mouth until said curved distal end surface abuts corniculate cartilage of the patient; and
pushing an endotracheal tube through said channel of said device.

18. The method of claim 17, further comprising at least one of steps:
connecting a ventilation mask to the proximal end of the device after said inserting step;
disconnecting the ventilation mask from the proximal end of the device after said connecting step and prior to said pushing step; and
coating at least a portion of a leading end of the endotracheal tube with a lubricant prior to said pushing step.

19. A method of inserting an endotracheal tube into a trachea of a patient, said method comprising the steps of:
inserting an airway device into the patient's mouth until a curved distal end surface of the device abuts corniculate cartilage of the patient, the device comprising:
a tubular member having a proximal end, a distal end opposite the proximal end, a tubular conduit positioned between the proximal end and the distal end, and a channel extending from a first channel opening at the proximal end through the tubular conduit to a second channel opening proximate the distal end; the distal end having an overall distal end width bound by opposing side walls, an overall distal end height bound by an uppermost distal end surface and a lower distal end surface, and a tear-drop shape represented by the uppermost distal end surface, the lower distal end surface, and the curved distal end surface connecting the uppermost distal end surface to the lower distal end surface, the curved distal end surface extending substantially perpendicular to and between the opposing side walls; and
pushing an endotracheal tube through the channel of the device.

20. The method of claim 19, wherein the device further comprises an epiglottis guard extending along an upper portion of the tubular member, the epiglottis guard comprising (i) a first end that is connected to the tubular member proximate the second channel opening, (ii) a second end that is not connected to the tubular member and positioned between the second channel opening and the distal end, and (iii) opposing edges extending from the first end to the second end, the opposing edges not connected to the tubular member,
wherein said pushing step causes the endotracheal tube to move the second end of the epiglottis guard away from the channel toward the patient's epiglottis so that the endotracheal tube enters the patient's trachea without interference from the patient's epiglottis.
